(19) United States
(12) Patent Application Publication
(10) Pub. No.: US 2010/0204258 A1
(43) Pub. Date: Aug. 12, 2010

(54) METHODS AND COMPOSITIONS FOR MODULATING INSULIN SECRETION AND GLUCOSE METABOLISM

(75) Inventors: Paul Harris, New York, NY (US); Yuli Xie, Shanghai (CN); Donald Landry, New York, NY (US); Shi-Xian Deng, White Plains, NY (US); Antonella Maffei, New York, NY (US)

Correspondence Address: BRYAN CAVE LLP, 1290 AVENUE OF THE AMERICAS, NEW YORK, NY, 10104, US

(73) Assignee: The Trustees of Columbia University in the City of New York, New York, NY (US)

(21) Appl. No.: 12/450,100

(22) PCT Filed: Mar. 12, 2008
(86) PCT No.: PCT/US08/03338

§ 371 Date: Apr. 26, 2010

Related U.S. Application Data
(60) Provisional application No. 60/906,623, filed on Mar. 12, 2007.
(60) Provisional application No. 60/932,810, filed on May 31, 2007.

(51) Int. Cl. A61K 31/4375; A61K 31/137; A61P 5/48; A61P 3/10

(52) U.S. Cl. 514/280; 514/294; 514/653

(57) ABSTRACT

The present invention relates to methods and compositions for treating or ameliorating the effects of diabetes. In addition, the present invention relates to methods and compositions for treating or preventing hyperglycemia, as well as modulating monoamine levels, islet β-cell insulin secretion, insulin and/or glucagon levels in a patient. In certain preferred embodiments, such methods include administering to a patient an effective amount of a vesicular monoamine transporter type 2 (VMAT2) antagonist, such as tetrabenazine (TBZ), dihydrotetrabenazine (DTBZ), tetrahydroberberine (THB), reserpine, emetine, Compound 6, or enantiomers, optical isomers, diastereomers, N-oxides, crystalline forms, hydrates, metabolites or pharmaceutically acceptable salts thereof.

### RELATED APPLICATIONS

This application is related to U.S. patent application Ser. No. 60/906,623, filed on Mar. 12, 2007, and Ser. No. 60/932,810, filed on May 31, 2007, which are incorporated herein in their entirety by reference.

### GOVERNMENT FUNDING

This invention was made with government support under Grant No. 5 R01 DK 63567 awarded by the National Institute of Diabetes & Digestive & Kidney Diseases. The government has certain rights in the invention.

### FIELD OF THE INVENTION

The field of the present invention relates to methods and compositions for treating or ameliorating the effects of diabetes. In addition, the present invention relates to methods and compositions for treating or preventing hyperglycemia, as well as modulating monoamine levels, islet β-cell insulin secretion, and insulin and glucagon levels in a patient.

### BACKGROUND OF THE INVENTION

D-Glucose, often in combination with certain amino acids, is the major physiological stimuli for insulin secretion. Net insulin production and glucose homeostasis, however, is regulated by a number of other substances, including several neurotransmitters that act directly on β-cells and indirectly though other target tissues. Many of these substances function as amplifying agents that have little or no effect by themselves, but enhance the signals triggered by the β-cell glucose sensing apparatus.

For example, during the cephalic phase of digestion, acetylcholine (ACh) is released via parasympathetic nerve terminals ending in islets. β-cells express the M3 muscarinic receptor and respond to exogenous ACh with increased inositol phosphate production, which in turn facilitates Na⁺ ion exit and calcium ion entry. This results in augmented insulin vesicle exocytosis. The amino acid glutamate, the major excitatory neurotransmitter in the central nervous system, can be found in both α- and β-cells of the endocrine pancreas. It is stored in glucagon- or insulin-containing granules, and appears to enhance insulin secretion when it is released. The presence of metabotropic glutamate receptors on α- and β-cells themselves suggests the presence of both autocrine and paracrine circuits within islet tissue involved in the regulation of insulin secretion.

Other neurotransmitters, such as the monoamines epinephrine and norepinephrine, released in circulation, may act to suppress glucose-stimulated insulin secretion by direct interaction with adrenoreceptors expressed (mainly the α-2 receptor) on pancreatic β-cells. β-cells of the endocrine pancreas also express dopamine receptors (D2) and respond to exogenous dopamine with inhibited glucose-stimulated insulin secretion. Purified islet tissue itself is a rich source of monoamines, and has been shown to contain 5-hydroxytryptamine, epinephrine, norepinephrine and dopamine.

β-cells also have the biosynthetic apparatus to create, dispose of, and store specific neurotransmitters. For example, islet tissue has been shown to include (a) tyrosine hydroxylase, the enzyme responsible for catalyzing the conversion of L-tyrosine to dihydroxyphenylalanine (DOPA), a precursor of dopamine, (b) L-DOPA decarboxylase, responsible for converting L-DOPA to dopamine, and (c) dopamine β-hydroxylase, the enzyme that catalyzes the conversion of dopamine to norepinephrine.

In addition, L-3,4-dihydroxyphenylalanine (L-DOPA) is rapidly converted to dopamine in islet β-cells. Monoamine oxidase (MAO) is a catabolic enzyme responsible for the oxidative de-amination of monoamines, such as dopamine and catecholamines, and maintains the homeostasis of monoamine-containing synaptic vesicles. The possible role of MAO in islet function has been studied, and MAO has been detected in the large majority of pancreatic islet cells, including β-cells. Interestingly, some MAO inhibitors have been shown to antagonize glucose-induced insulin secretion. The secretory granules of pancreatic β-cells have been documented to have the ability to store substantial amounts of calcium, dopamine, and serotonin therein.

In the central nervous system, the storage of monoamine neurotransmitters in secretory organelles is mediated by vesicular amine transporters. These molecules are expressed as integral membrane proteins of the lipid bilayer of secretory vesicles in neuronal and endocrine cells. By way of an electrochemical gradient, the vesicular amine transporters exchange one cytosolic monoamine, such as dopamine, for two intravesicular protons functioning to package neurotransmitters for later discharge into the synaptic space. Both immunohistochemistry and gene expression studies have shown that islet tissue and the β-cells of the endocrine pancreas selectively express only one member of the family of vesicular amine transporters, namely, vesicular monoamine transporter type 2 (VMAT2).

Recent studies have examined the feasibility of noninvasive measurements of the amount of VMAT2 in the pancreas using its specific radioligand [¹¹C] DTBZ (dihydrotetrabenazine) and positron emission tomography as a surrogate measure of β-cell mass, but the possible role of VMAT2 (as expressed in islet tissue and β-cells) in glucose metabolism has not yet been explored. Substantial evidence, as partially outlined above, suggests that endogenously synthesized and/or stored monoamine neurotransmitters participate in paracrine regulation of insulin secretion and entrainment of the activity of the different cell populations within islets.

Given the important role of vesicular amine transporters in the storage and distribution of such monoamine neurotransmitters, there is a need for methods and compositions that could be used to effectively modulate the activity of such transporters, such as VMAT2. Such methods and compositions may be used, for example, to regulate insulin production, achieve glucose homeostasis, and/or treat or ameliorate the effects of diabetes.

### SUMMARY OF THE INVENTION

According to a first preferred embodiment of the invention, methods are provided for treating or ameliorating the effects of diabetes. Such methods comprise administering to a patient an effective amount of a vesicular monoamine transporter type 2 (VMAT2) antagonist. In certain embodiments, such methods may comprise intravenously administering to a patient in need thereof about 1.6 mg/kg body weight of a VMAT2 antagonist selected from the group consisting of tetrabenazine (TBZ), dihydrotetrabenazine (DTBZ), and enantiomers, optical isomers, diastereomers, N-oxides, crystalline forms, hydrates, metabolites, and pharmaceutically acceptable salts thereof. In other embodiments, such methods may comprise intravenously administering to a patient in need thereof about 2 mg/kg body weight of a VMAT2 antagonist selected from the group consisting of tetrahydroberberine (THB), reserpine, emetine, Compound 6, or enantiomers, optical isomers, diastereomers, N-oxides, crystalline forms, hydrates, metabolites, pharmaceutically acceptable salts, or combinations thereof.

According to another preferred embodiment of the invention, methods are provided for treating or preventing hyperglycemia, which comprises administering to the patient an effective amount of a VMAT2 antagonist. In certain embodiments, such methods may comprise intravenously administering to a patient in need thereof about 1.6 mg/kg body weight of a VMAT2 antagonist selected from the group consisting of TBZ, DTBZ, and enantiomers, optical isomers, diastereomers, N-oxides, crystalline forms, hydrates, metabolites, and pharmaceutically acceptable salts thereof. In other embodiments, such methods may comprise intravenously administering to a patient in need thereof about 2 mg/kg body weight of a VMAT2 antagonist selected from the group consisting of tetrahydroberberine (THB), reserpine, emetine, Compound 6, or enantiomers, optical isomers, diastereomers, N-oxides, crystalline forms, hydrates, metabolites, pharmaceutically acceptable salts, or combinations thereof.

According to further embodiments of the present invention, methods for modulating monoamine levels or, such as, e.g., depleting monoamine levels from a patient's pancreas are provided, wherein monoamine levels in such patient's brain are not significantly altered. In addition, the present invention provides methods for modulating islet β-cell insulin secretion and insulin and glucagon levels, and for regulating insulin production and glucose homeostasis in a patient in need of such modulation or regulation. In such embodiments, the methods comprise administering to the patient an effective amount of a VMAT2 antagonist.

According to still further embodiments of the invention, methods for modulating glucose-stimulated insulin secretion in human islets are provided. Such methods comprise providing to the islets an amount of a VMAT2 antagonist that is effective to achieve such modulation.

### BRIEF DESCRIPTION OF THE FIGURES

FIG. 1. Tetrabenazine (TBZ) reduces the blood glucose excursion during an intraperitoneal glucose tolerance test (IPGTT). Left panel. Blood glucose values during an IPGTT of Lewis rats (9-11 week old) treated with vehicle alone (open symbols) or with tetrabenazine (1.6 μg/gm body weight) (closed symbols). Right panel. Cumulative results from a series of experiments (n=4). The AUC (area under the curve) IPGTT for controls was significantly higher than the AUC IPGTT TBZ treated animals (p<0.05). Error bars represent the standard error of the mean.

FIG. 2. TBZ reduces the blood glucose excursion in a dose dependent manner. Area under the curve from glucose tolerance tests (AUCIPGTT) of Lewis rats treated with varying doses of tetrabenazine. A baseline untreated IPGTT was determined for each animal. One week later, a second IPGTT was performed with varying doses of TBZ. Two or more animals were used at each dose level. The area under the curve was calculated for each test and the results for TBZ-treated animals were normalized to their respective baseline measurement. Results are presented as the mean of two or more measurements and the error bars indicate the highest and lowest measurement at the indicated dose.

FIG. 3. TBZ reduces the dopamine content of brain and pancreas tissue. TBZ at 1.6 μg/gm body weight was administered intravenously to Lewis rats. One hour later, the animals were euthanized and the brains and pancreata harvested and extracted in buffer. The dopamine concentration in the extract was determined by ELISA and normalized to the total protein content.

FIG. 4. TBZ reduces the blood glucose excursion during IPGTT in diabetic Lewis rats. Blood glucose values during an IPGTT of Lewis rats (5-7 weeks old) were measured before treatment with streptozotocin (open circle) and following induction of diabetes with streptozotocin (triangles). The IPGTT response was first measured in diabetic rats treated with TBZ (1.6 μg/gm) (closed triangles) and then several days later with vehicle alone (open triangles). Data from a representative experiment in a series of three animals. Inset. The abundance of insulin transcripts in the pancreas of streptozotocin (STZ)-treated animals used in these experiments was measured after IPGTT testing and compared to the mean transcript abundance of a group of three control animals. Error bars represent the standard error of the mean.

FIG. 5. Quantitation of VMAT2 protein in pancreas of control and STZ-treated Lewis rats. Pancreata were removed en block from control and diabetic Lewis rats and solubilized in SDS page buffer with protease inhibitor cocktail. Lysates were separated in the first dimension by SDS page. Proteins were then transferred electrophoretically to membranes, blocked and probed with either anti-VMAT2 or anti-insulin antibodies. The bands were then developed with a HRP-conjugated secondary antibody and chemiluminescent substrate solution.

FIG. 6. TBZ alters glucose-stimulated insulin and glucagon secretion in vivo. Serum insulin (B) and glucagon (C) concentrations and blood glucose concentrations (A) were measured during IPGTT of Lewis rats (9-11 week old) treated with vehicle alone (open symbols) or with TBZ (1.6 μg/gm) (closed symbols). Data from a representative experiment in a series of three animals were tested. Measurements are means and standard errors from triplicate determinations of serum/blood samples.

FIG. 7. Dihydrotetrabenazine (DTBZ) enhances glucose-stimulated insulin secretion in human islets ex vivo. Purified cadaveric islets were cultured in high or low glucose-containing media with and without DTBZ and epinephrine. During the incubation period, the insulin secretion rate (ISR) of the islets was determined by ELISA.

FIG. 8. VMAT2 localizes to human islets in situ. Human cadaveric pancreas tissue was processed for immunohistochemistry and probed with anti-VMAT2 antibodies. The pattern of staining is limited to the central islet of Langerhans and an occasional nerve fiber.

FIG. 9. A diagram showing the effect of TBZ on glucose homeostasis.

FIG. 10. TBZ, tetrahydroberberine (THB), reserpine, and emetine reduce the blood glucose excursion during an intraperitoneal glucose tolerance test (IPGTT). Butamol does not reduce the blood glucose excursion during an IPGTT. Blood glucose values during an IPGTT of Lewis rats (9-11 weeks old) treated with 2 mg/kg body weight of vehicle (dimethyl sulfoxide (DMSO)) alone (diamonds), TBZ (lighter squares), THB (triangles), butamol (darker squares) reserpine (larger circles), and emetine (smaller circles) are shown.

FIG. 11. A diagram showing synthetic schemes for Compound 6.

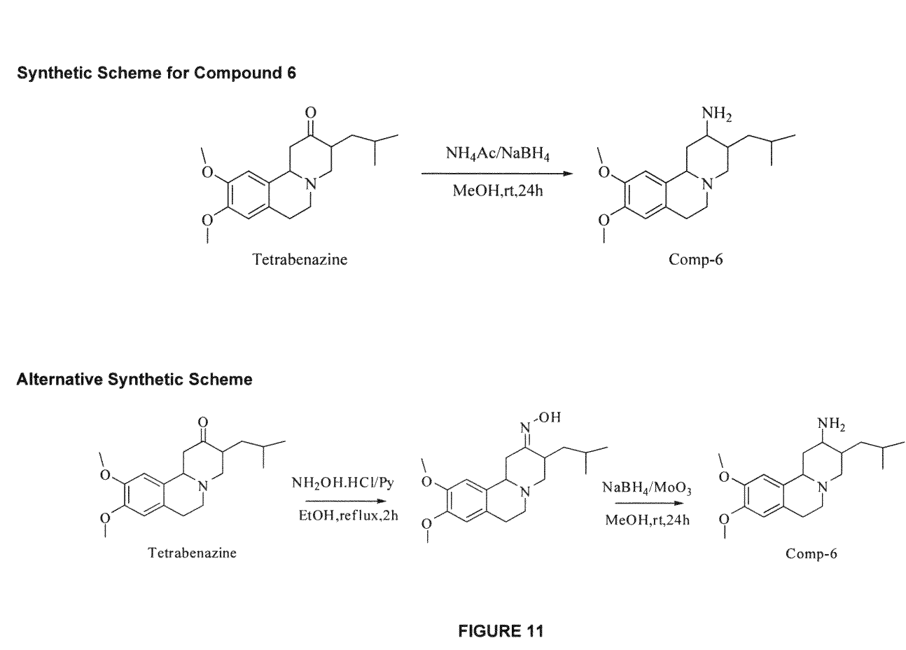

FIG. 12. TBZ, emitine, and Compound 6 depress the area under the curve from glucose tolerance tests. Each series is a separate experiment.

### DETAILED DESCRIPTION OF THE INVENTION

According to a first preferred embodiment of the invention, methods are provided for treating or ameliorating the effects of diabetes. Such methods comprise administering to a patient an effective amount of a vesicular monoamine transporter type 2 (VMAT2) antagonist. In certain embodiments, such methods may comprise intravenously administering to a patient in need of such treatment, e.g., a diabetic patient, about 1.6 mg/kg body weight of a VMAT2 antagonist. The antagonist is preferably tetrabenazine (TBZ), dihydrotetrabenazine (DTBZ), or enantiomers, optical isomers, diastereomers, N-oxides, crystalline forms, hydrates, metabolites, or pharmaceutically acceptable salts thereof. In the present invention, combinations of one or more of TBZ, DTBZ and their respective enantiomers, optical isomers, diastereomers, N-oxides, crystalline forms, hydrates, metabolites, and pharmaceutically acceptable salts are also contemplated. In other embodiments, such methods may comprise intravenously administering to a patient in need thereof about 2 mg/kg body weight of a VMAT2 antagonist. The antagonist is preferably tetrahydroberberine (THB), reserpine, emetine, Compound 6, or enantiomers, optical isomers, diastereomers, N-oxides, crystalline forms, hydrates, metabolites, pharmaceutically acceptable salts, or combinations thereof.

According to another preferred embodiment of the invention, methods are provided for treating or preventing hyperglycemia, which comprises administering to a patient an effective amount of a VMAT2 antagonist. In certain embodiments, such methods may comprise intravenously administering to a patient in need thereof, e.g., a hyperglycemic patient, about 1.6 mg/kg body weight of a VMAT2 antagonist. The antagonist is preferably TBZ, DTBZ, or enantiomers, optical isomers, diastereomers, N-oxides, crystalline forms, hydrates, metabolites, or pharmaceutically acceptable salts thereof. In other embodiments, such methods may comprise intravenously administering to a patient in need thereof about 2 mg/kg body weight of a VMAT2 antagonist. The antagonist is preferably THB, reserpine, emetine, Compound 6, or enantiomers, optical isomers, diastereomers, N-oxides, crystalline forms, hydrates, metabolites, pharmaceutically acceptable salts, or combinations thereof.

According to further embodiments of the present invention, methods for modulating monoamine levels (e.g., dopamine levels) are provided. Such methods comprise administering to a patient in need of such modulation an effective amount of a VMAT2 antagonist. More specifically, the present invention provides methods of depleting monoamine levels in a patient's pancreas, without substantially altering the monoamine levels in such patient's brain. Still further, the present invention provides methods for modulating islet β-cell insulin secretion and insulin and glucagon levels, and for regulating insulin production and glucose homeostasis in a patient in need of such modulation or regulation. In such embodiments, the methods comprise administering to the patient an effective amount of a VMAT2 antagonist, such as TBZ, DTBZ, THB, reserpine, emetine, Compound 6, or enantiomers, optical isomers, diastereomers, N-oxides, crystalline forms, hydrates, metabolites, pharmaceutically acceptable salts, or combinations thereof.

As used herein in relation to monoamine levels, islet β-cell insulin secretion, and insulin and glucagon levels, “modulate,” “modulating,” and like terms mean to increase or decrease the monoamine, islet β-cell insulin secretion, and/or insulin and glucagon levels in a mammal, e.g., a human patient administered a VMAT2 antagonist according to the present invention relative to a patient who is not administered the VMAT2 antagonist. Preferably, with respect to monoamine levels, “modulating” means to decrease the monoamine, e.g., dopamine, levels in a patient, more preferably to lower the monoamine levels in the pancreas without affecting the monoamine levels in the brain.

With respect to islet β-cell insulin secretion, “modulating” means to increase (3-cell insulin secretion in a patient administered a VMAT2 antagonist according to the present invention relative to a patient who is not administered the VMAT2 antagonist. With respect to insulin and glucagon levels, “modulating” means to increase plasma insulin levels and decrease plasma glucagon levels in a patient administered a VMAT2 antagonist according to the present invention compared to a patient not treated with the VMAT2 antagonist.

As used herein in relation to insulin production and glucose homeostasis, “regulate,” “regulating,” or like terms mean to exert control of those processes through administration of a VMAT2 antagonist to a patient whose insulin production and/or glucose levels deviate from a normal clinical value.

In the Examples, representative methods for determining monoamine levels, islet β-cell insulin secretion levels, insulin and glucagon levels, and insulin production and blood/serum glucose levels in, e.g., a human patient are described. The present invention, however, embraces any art-recognized method for making such determinations. For example, a patient's blood glucose (BG) levels may be monitored and/or determined using an Accu-Check blood glucose monitoring system (Roche Diagnostics, Sommerville, N.J.).

According to still further embodiments of the invention, methods for modulating glucose-stimulated insulin secretion in human islets are provided. Such methods comprise providing to the islets an amount of a VMAT2 antagonist that is effective to achieve such modulation. Such a VMAT2 antagonist may be selected from TBZ, DTBZ, or enantiomers, optical isomers, diastereomers, N-oxides, crystalline forms, hydrates, metabolites, or pharmaceutically acceptable salts thereof.

In the present invention, an “effective amount” or “therapeutically effective amount” of a VMAT2 antagonist is an amount of such an antagonist that is sufficient to effect beneficial or desired results as described herein. In terms of treatment of a mammal, e.g., a human patient, an “effective amount of a VMAT2 antagonist” is an amount sufficient to treat, manage, palliate, ameliorate, or stabilize a condition, such as diabetes (including type-1 or type-2) or hyperglycemia, in the mammal.

In the present invention, an effective amount of a VMAT2 antagonist will be sufficient to reduce or deplete monoamine levels from a patient's pancreas, but not effect monoamine levels in the patient's brain. Typically, in the present invention, an effective amount of a VMAT2 antagonist is between about 0.2 mg/kg body weight to about 5.0 mg/kg body weight of the VMAT2 antagonist or, preferably, 0.5 to about 3.3 mg/kg body weight, such as 1.6 mg/kg body weight or 2 mg/kg body weight. In the present invention, the foregoing amounts may be provided to a patient for the desired treatment course. Preferably, during a course of treatment, no more than about 3.3 mg of a VMAT2 antagonist is administered.

In the present invention, when a range is stated for a particular parameter, e.g., an effective amount, all values within that range, including the endpoints, are intended to be included. In addition to the foregoing, effective dosage forms, modes of administration, and dosage amounts of the VMAT2 antagonists may be determined empirically, and making such determinations is within the skill of the art in view of the disclosure herein. It is understood by those skilled in the art that the dosage amount will vary with the route of administration, the rate of excretion, the duration of the treatment, the identity of any other drugs being administered, the age, size, and species of mammal, and like factors well known in the arts of medicine and veterinary medicine. In general, a suitable dose of a VMAT2 antagonist according to the invention will be that amount of the VMAT2 antagonist, which is the lowest dose effective to produce the desired effect. The effective dose of a VMAT2 antagonist maybe administered as one, two, three, four, five, six or more sub-doses, administered separately at appropriate intervals throughout the day.

A VMAT2 antagonist of the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. In the present invention, a preferred route of administration is intravenous. Further, a VMAT2 antagonist of the present invention may be administered in conjunction with other treatments. A VMAT2 antagonist or composition containing such an antagonist may be encapsulated or otherwise protected against gastric or other secretions, if desired.

While it is possible for a VMAT2 antagonist of the invention to be administered alone, it is preferable to administer the VMAT2 antagonist as a pharmaceutical formulation (composition). Pharmaceutically acceptable compositions of the invention comprise one or more VMAT2 antagonists as an active ingredient in admixture with one or more pharmaceutically-acceptable carriers and, optionally, one or more other compounds, drugs, ingredients and/or materials. Regardless of the route of administration selected, the VMAT2 antagonists of the present invention are formulated into pharmaceutically-acceptable dosage forms by conventional methods known to those of skill in the art. See, e.g., Remington's Pharmaceutical Sciences (Mack Publishing Co., Easton, Pa.).

Pharmaceutically acceptable carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active VMAT2 antagonist. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active VMAT2 antagonist may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more VMAT2 antagonist in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, and/or solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods and compositions of the present invention. These examples are illustrative only and are not intended to limit the scope of the invention in any way.

### EXAMPLES

The following examples demonstrate, inter alia, that a single in vivo administration of tetrabenazine (TBZ) to control and streptozotocin (STZ)-treated Lewis rats results in enhanced glucose-stimulated insulin secretion and a smaller glucose excursion following intraperitoneal glucose tolerance testing. The following also demonstrates that in vivo administration of TBZ depletes the dopamine content of pancreas tissues. In the in vitro studies described below, it is further demonstrated that dihydrotetrabenazine (DTBZ), the direct and active metabolite of TBZ, enhances glucose-stimulated insulin secretion by purified human cadaveric islets. Together, the examples show, inter alia, that VMAT2 expressed within the tissue of the endocrine pancreas has an important role in the regulation of insulin production and glucose homeostasis in vivo and, moreover, constitutes a new target for therapeutic intervention of insulin-related diseases, such as diabetes.

### Example 1

### Materials and Methods

Drugs and reagents. L-epinephrine bitartrate, STZ, D-glucose, and sodium citrate were obtained from Sigma Chemical Company (St. Louis, Mo.). All cell culture media and supplements were obtained from Invitrogen (Carlsbad, Calif.). Tissue culture plates were obtained from Falconware (Becton-Dickinson, Inc., Oxnard, Calif.). Tetrabenazine and dihydrotetrabenazine were obtained from the National Institute of Mental Health's Chemical Synthesis and Drug Supply Program.

Experimental animals. All animal studies were reviewed and approved by the Institutional Animal Care and Use Committee (IACUC) at Columbia University's Medical School. All experiments were performed in accordance with the IACUC approved procedures. Normal male Lewis rats (100-400 grams) were obtained from Taconic (Taconic Inc., Germantown, N.Y.) and were housed under conditions of controlled humidity (55±5%), temperature (23±1° C.), and lighting (light on: 06.00-18.00 hours) with free access to standard laboratory rat chow and water. Rats were handled daily to minimize nonspecific stress for more than 7 days before the experiments began. In most experiments, it was necessary to measure blood glucose in fasting animals. For these groups, food was removed at the beginning of the light cycle, 6 hours before glucose levels were measured. Diabetes mellitus was induced by intraperitoneal injection of streptozotocin (Sigma Chemical Co., St. Louis, Mo.) (25 to 50 mg/kg) to animals (100 to 150 grams) that had been fasted 4 hours to enhance the effectiveness of STZ treatment.

The STZ solution was prepared fresh by dissolving it in 0.1 M citrate buffer (pH 5.5) and terminally sterile filtered. Control Lewis age and weight matched rats received a 0.5 ml/kg citrate vehicle alone via intraperitoneal injection. Sixty minutes prior to intraperitoneal glucose tolerance testing (IPGTT), anesthesia of male Lewis rats was induced with isoflurane (3-4% in oxygen) and maintained with 1-2% isoflurane in oxygen. Anaesthetized rats were administered TBZ at the indicated dose by intravenous (i.v.) injection using the penile vein. TBZ was dissolved in neat sterile dimethylsulfoxide (DMSO) and diluted (always more than 10 fold) in sterile saline. Rats received injections of vehicle alone (10% DMSO in saline) or reserpine (in saline). Animals recovered fully before receiving IPGTT.

Blood glucose, insulin, glucagon and intraperitoneal glucose tolerance tests measurements. Blood samples were collected from a superficial blood vessel in the tails of the rats following 6 hours of fasting between 12:00 noon and 2:00 p.m. The fasting blood glucose (BG) levels of the rats were measured using an Accu-Check blood glucose monitoring system (Roche Diagnostics, Sommerville, N.J.). Intraperitoneal glucose tolerance tests (IPGTT) were performed in 6 hour fasting un-anaesthetized animals. Briefly, after baseline BG measurements, animals received an intraperitoneal (i.p.) injection of 1 gram glucose/kilogram body weight. To minimize stress during the procedure, rats were handled by the same operator during acclimatization and later during weighing and IPGTT. Blood samples (approximately 30 μl) were collected at baseline and then again 15, 30, 60, 90, and 120 minutes following i.p. glucose administration. BG concentrations were measured immediately on these samples and the remainder processed.

Plasma was immediately separated by centrifugation at 3000×g for 15 minutes and then stored at −20° C. until analysis. Insulin and glucagon concentration measurements in rat plasma were performed by ELISA as per the manufacturer's instructions using kits from Linco Research Inc. (St. Charles, Mo.) and Alpco Diagnostics (Salem, N.H.), respectively. To validate the test, saline injections were performed by the same method. During this experiment, glucose concentration did not differ from baseline at each time point (data not shown). The area under the IPGTT glucose concentration×time curve (AUCIPGTT) was calculated by the trapezoidal rule. The area under the insulin or glucagon concentration×time curve (AUC INS or AUC GCG) was calculated in a similar manner. For Lewis rats receiving STZ, the animals were considered diabetic when they showed abnormal IPGTT responses and fasting BG values above about 300 mg/dL on two or more occasions.

Human islet tissue and glucose-stimulated insulin secretion. The islet tissue used in these studies was obtained with institutional review board approval. Pancreas digestion and islet isolation were performed using minor modifications of the Edmonton purification protocol. (Shapiro, A. M., J. Lakey et al. “Islet preparation in seven patients with type I diabetes mellitus using a glucocortoid-free immunosuppressive regimen.” New England Journal of Medicine 343(4):230-8, (2000)) The determination of islet cell mass, viability, and purity were also performed. Purified islets were cultured in CMRL 1066 culture media with 10% fetal bovine serum at 37° C. in humidified air (5% CO₂) for 18 to 24 hours. The human islet insulin secretory response was performed according to a procedure described by the Edmonton group. (Id.) Briefly, after an overnight culture, islets were incubated with either low or high concentrations of glucose for 2 hours at 37° C. and 5% CO₂. The supernatant was collected for insulin measurement. Insulin concentrations in these experiments were analyzed with a human insulin enzyme-linked immunosorbent assay (ELISA) kit (ALPCO Insulin ELISA kit, Windham, N.H.). In some experiments TBZ, DTBZ or epinephrine was added to the cultures before glucose stimulation.

Dopamine measurements. Anaesthetized rats received an intravenous injection of TBZ and were sacrificed one hour later. Euthanasia was performed by exsanguination of the anesthetized animal. Brain and pancreas were harvested as quickly as possible and frozen at −80° C. until use. Frozen tissue was pulverized in a liquid nitrogen cooled mortar and extracted in 0.01 N HCl. The tissue extract was centrifuged at 10,000×g at 4° C. to remove debris and the total protein was estimated by reading the absorbance at 280 nm. The concentration of dopamine in the extract was estimated using an ELISA kit from Rocky Mountain Diagnostics (Colorado Springs, Colo.) per the manufacturer's instructions and normalized to the extract protein concentration.

Quantitation of VMAT2 and proinsulin transcript abundance in pancreata of Lewis rats. Harvesting of pancreata was performed by opening the anesthetized rats with a midline incision and reflecting the liver, stomach and small intestines to expose the pancreas. The cavity was then bathed with 5 ml of RNAlater (Ambion, Austin, Tex.) per the manufacturer's recommendations. The head, body and tail of the pancreas were dissected under RNAlater and removed to a 25 mm plastic Petri dish containing sufficient RNAlater to cover the excised tissue. The pancreas was cut into approximately 2×2×2 mm sections and transferred to fresh RNAlater and stored overnight at 4° C. Total pancreatic RNA was isolated and specific transcript abundances were measured by real-time quantitative RT-PCR. The conditions used were as follows: one cycle at 95° C. for 900 seconds followed by 45 cycles of amplification (94° C. for 15 seconds, 55° C. for 20 seconds, and 72° C. for 20 seconds). The oligonucleotides were synthesized by Invitrogen. The primer sequences used were as follows:

(SEQ ID NO: 1)

5′-CTTCGACATCACGGCTGATGG-3′(Cyclophilin A-5′)
and

(SEQ ID NO: 2)

5′-CAGGACCTGTATGCTTCAGG-3′(Cyclophilin A-3′),

(SEQ ID NO: 3)

5′-GCC CTG CCC ATC TGG ATG AT-3′(VMAT2-5′)
and

(SEQ ID NO: 4)

5′-CTT TGC AAT AGC ACC ACC AGC AG-3′(VMAT2-3′),

(SEQ ID NO: 5)

5′-CCC AGG CTT TTG TCA AAC-3′(rINS1/2 - 5′)
and

(SEQ ID NO: 6)

5′-CTT GCG GGT CCT CCA CTT 3′(rINS1/2 - 3′).

The relative amounts of mRNA were calculated by the comparative cycle threshold (CT) method. Such values were then normalized by cyclophilin A expression.

Quantitation of VMAT2 and insulin protein in pancreas lysates by Western blot. Western blot analysis was conducted of brain and pancreas tissue obtained from control and diabetic streptozotocin treated rats. Briefly, sample tissue were prepared in RIPA buffer (1×PBS; 1% Igepal CA-630; 0.5% sodium deoxycholate; 0.1% SDS; 10 mg/ml complete protease inhibitor cocktail (Roche Inc, Palo Alto, Calif.)) at 4° C. Protein concentrations were determined using a Bio-Rad protein assay (Bio-Rad Inc., Hercules, Calif.). Protein separation and gel transfer were carried out using the NuPage/Novex XCELLII system for 4-12% gradient Bis-Tris gels and MOPS running buffer (Invitrogen, Carlsbad, Calif.). After transfer, PVDF membranes were washed in Tris-Buffered Saline (TBS), blocked in TBS-5% non-fat milk and incubated with a rabbit anti-hVMAT2-Ct primary antibody (Chemicon, Temecula, Calif.) or anti-insulin primary antibody (Phoenix Pharmaceuticals, Burlingame, Calif.) at 1:1000 in TBS-T (TBS, 0.075% Tween-20) overnight at 4° C. The membranes were washed in TBS-T and incubated with a goat anti-rabbit secondary antibody conjugated with horseradish peroxidase (HRP) (Santa Cruz Biotechnology, Santa Cruz, Calif.) at 1:3333 in TBS-T for 1 hour at room temperature and washed again in TBS-T. The membranes were placed in West Pico chemiluminescent solution (Pierce, Rockford, Ill.) and developed on a FujiFilm developer.

Immunohistochemistry. Cadaveric pancreas tissue was fixed and paraffin embedded by standard methods. Sections were deparaffinized with a series of graded alcohols and xylenes. Antigen retrieval was achieved by microwave treatment with 10 mM sodium citrate (pH 6) for 10 minutes. Endogenous peroxidase was quenched with a 3% hydrogen peroxide solution for 20 minutes. Sections were then blocked with CAS Block (Zymed, San Francisco, Calif.) followed by incubations with (1) anti-VMAT2 primary antibody overnight at 4° C. (1:200, Chemicon); (2) biotinylated goat anti-rabbit IgG secondary antibody (1:200, Vector, Burlingame, Calif.) for 1 hour at room temperature; and (3) HRP-Streptavidin (Zymed) for 1 hour at room temperature. Color was then developed with an enhanced DAB kit (Abcam, Cambridge, Mass.) and sections were lightly counterstained with hematoxylin (Vector).

Statistical Analysis. All results are presented as means±SEM or as indicated in the text. Statistical strength of associations was estimated by the method of Student t-testing.

### Example 2

### Materials and Methods

Drugs and reagents. Tetrabenazine, tetrahydroberberine (THB), butamol, reserpine, and emetine are commercially available or are obtained from the National Institute of Mental Health's Chemical Synthesis and Drug Supply Program. Compound 6 (3-isobutyl-9,10-dimethoxy-2,3,4,6,7,11b-hexahydro-1H-pyrido[2,1-a]isoquinolin-2-amine) was synthesized as described below.

### Synthesis of Compound-6

Tetrabenazine (317 mg, 1 mmol) was dissolved in methanol (MeOH, 10 ml) and cooled with ice-water. To this solution, ammonia acetate (500 mg) was added, followed by the addition of sodium borohydride (50 mg) in portion. The reaction was stirred at room temperature for 24 hours and quenched with water. The aqueous solution was extracted with methylene chloride (10 ml) twice. The combined organic phase was washed with brine and dried with sodium sulfate. After removing the solvent, the residue was purified by chromatography. One hundred and fifty milligrams of Compound 6 (3-isobutyl-9,10-dimethoxy-2,3,4,6,7,11b-hexahydro-1H-pyrido[2,1-a]isoquinolin-2-amine) was obtained as white solid (yield: 47%).

Compound 6 was also synthesized using the following alternative method: tetrabenazine (317 mg, 1 mmol) was dissolved in ethanol (EtOH, 10 ml) and hydroxylamine hydrochloride (70 mg, 1 mmol) was added, followed by the addition of pyridine (1 ml). The reaction was refluxed for 2 hours. After removing solvent, the residue was redissolved in methanol (MeOH, 10 ml). To this solution, MoO₃ (80 mg) and sodium borohydride (80 mg) were slowly added. The reaction was stirred at room temperature for 24 hours and quenched with water. The aqueous solution was extracted with methylene chloride (10 ml) twice. The combined organic phase was washed with brine and dried with sodium sulfate. After removing solvent, the residue was purified by chromatography. Two hundred and fifty milligrams of Compound 6 (3-isobutyl-9,10-dimethoxy-2,3,4,6,7,11b-hexahydro-1H-pyrido[2,1-a]isoquinolin-2-amine) was obtained as white solid (yield: 78%).

The structures of THB, butamol, reserpine, emetine, and Compound 6 are shown below:

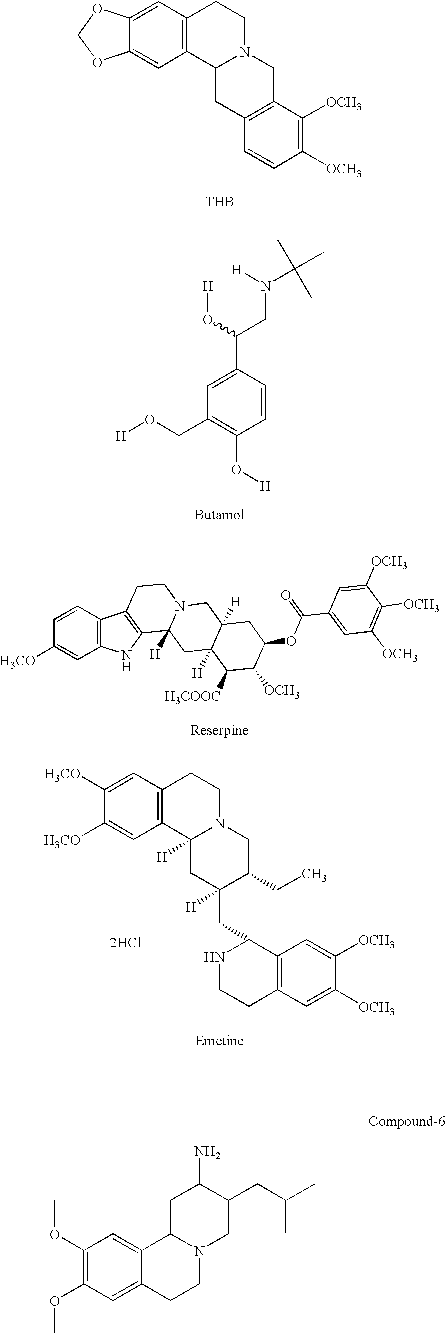

Experimental animals. All animal studies were conducted as described in Example 1.

Anaesthetized rats were administered TBZ, THB, butamol, reserpine, emetine, or Compound 6 at a dose of approximately 2-3 mg/kg body weight by intravenous (i.v.) injection using the penile vein. TBZ, THB, butamol, reserpine, emetine, and Compound 6 were each separately dissolved in neat sterile dimethylsulfoxide (DMSO) and diluted (always more than 10 fold) in sterile saline. Rats received injections of vehicle alone (10% DMSO in saline) or reserpine (in saline). Animals recovered fully before receiving IPGTT.

Blood glucose, insulin, glucagon and intraperitoneal glucose tolerance tests measurements. Blood samples were collected from a superficial blood vessel in the tails of the rats following 6 hours of fasting between 12:00 noon and 2:00 p.m. The fasting blood glucose (BG) levels of the rats were measured using an Accu-Check blood glucose monitoring system (Roche Diagnostics, Sommerville, N.J.). Intraperitoneal glucose tolerance tests (IPGTT) were performed in 6 hour fasting un-anaesthetized animals. Briefly, after baseline BG measurements, animals received an intraperitoneal (i.p.) injection of 1 gram glucose/kilogram body weight. To minimize stress during the procedure, rats were handled by the same operator during acclimatization and later during weighing and IPGTT. Blood samples (approximately 30 μl) were collected at baseline and then again 15, 30, 45, 60, 90, and 120 minutes following i.p. glucose. BG concentrations were measured immediately on these samples and the remainder processed.

### Example 3

### Results & Analysis

Glucose tolerance in Lewis rats is improved by TBZ. Older Lewis rats have a relative glucose intolerance compared to younger animals during an IPGTT. To explore the role of VMAT2 in insulin secretion and to better demonstrate the possible value of VMAT2 as a potential therapeutic target in diabetes, older male Lewis rats were selected for IPGTT testing with and without a single dose of tetrabenazine. A dose of tetrabenazine approximately three to ten fold higher than the equivalent human doses currently used to treat movement disorders was used in this example. Following TBZ administration, but before glucose challenge, no reproducible differences were observed in the baseline fasting glucose concentration of control animals (data not shown).

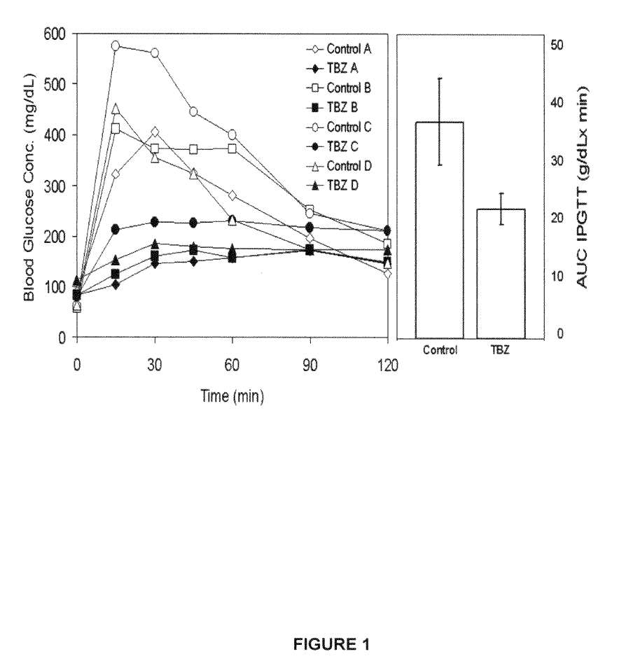

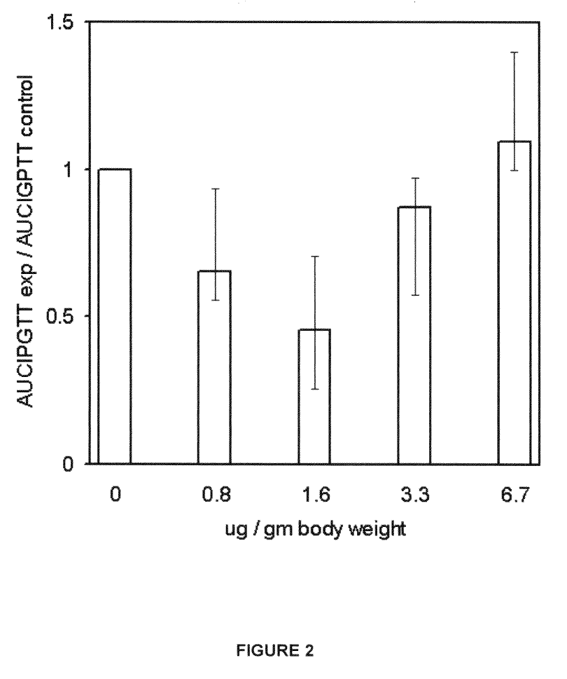

Following tetrabenazine treatment and glucose challenge, however, a significant change in the size and shape of the glucose disposition curve was observed during IPGTT (FIG. 1). For example, the characteristic rise in glucose concentration around 15 minutes after injection was blunted following TBZ administration. A comparison of the areas under the curve during IPGTT reveals that TBZ reduced the glucose excursion by 40-50% at 1.6 μg/gbm (gram body weight). When the dose of TBZ given before IPGTT was varied, a complex dose response effect resulted (FIG. 2).

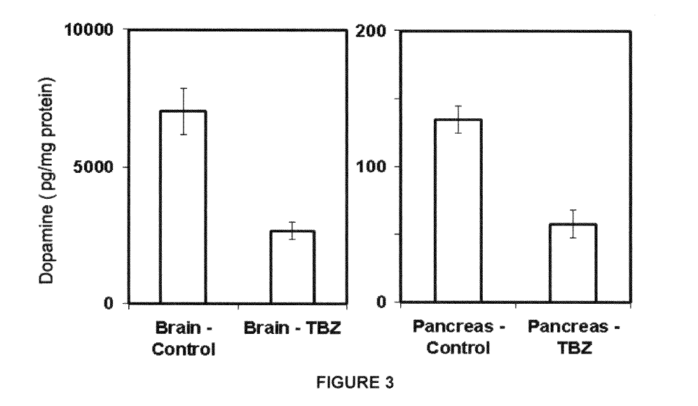

Because reserpine also binds to VMAT2, albeit with a higher dissociation constant (but with less selectivity), the effects of a single concentration of reserpine (25 μg/gbm) in the Lewis IPGTT was also tested. It was found that reserpine induced a persistent hyperglycemia and larger AUC IPGTT relative to the untreated controls (data not shown). It is known that tetrabenazine will reduce the concentration of monoamines in the CNS, and that dopamine is a well-known substrate of VMAT2-mediated vesicular transport. Thus, the effects of tetrabenazine on the concentration of the monoamine dopamine in both the pancreas and brain was tested one hour after injection of 10 μg/gm body weight of tetrabenazine. This test demonstrated that TBZ significantly depleted the dopamine content of pancreas and brain (FIG. 3).

Glucose tolerance in Diabetic Lewis rats is improved by TBZ. Whether the glucose tolerance enhancing effects of TBZ might extend to animals with reduced β-cell mass and impaired glucose tolerance due to STZ-induced diabetes was next examined. For these experiments, younger animals (5-8 weeks of age) were selected—for their better tolerance of induction of diabetes with STZ. From a pool of animals treated with streptozotocin, rats that showed high fasting glucose concentrations and impaired glucose tolerance were selected, which were characterized by high early glucose levels (>300 mg/dl) that peaked and gradually diminished (but did not return to baseline levels within the duration of the two hour IPGTT test).

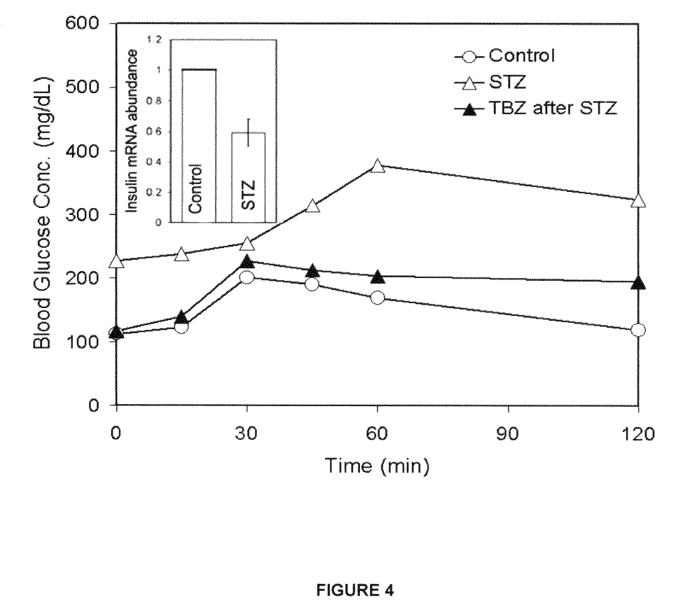

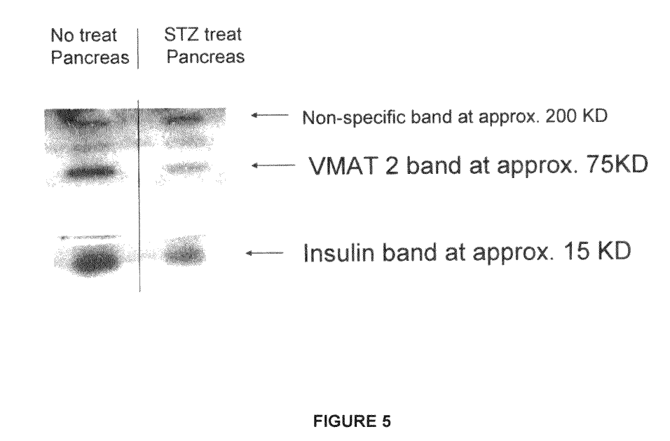

During IPGTT testing, blood glucose levels were returned to control or near normal levels at around sixty minutes following i.v. injection of TBZ, but before i.p. glucose challenge (FIG. 4). Following glucose challenge, and similar to normal Lewis rats treated with TBZ, it was found that TBZ administration resulted in a smaller area under the curve in the IPGTT. The glucose tolerance-enhancing effects of TBZ were not observed if the selected TBZ-untreated animals had initial AUC IPGTT>50,000 minutes×mg/dl (data not shown). The loss of insulin within the endocrine pancreas following STZ treatment was validated by quantitative RT-PCR (FIG. 4 inset). The loss of VMAT2 protein within the pancreas following STZ treatment was also validated by western blotting (FIG. 5).

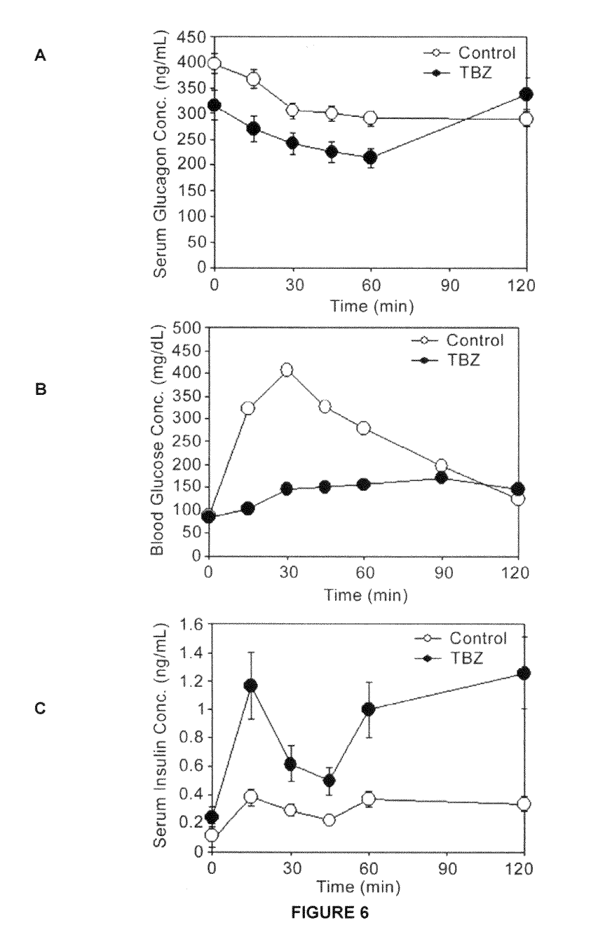

TBZ enhances in vivo and in vitro glucose dependent insulin secretion. Whether the smaller glucose excursions in IPGTT seen after administration of TBZ were due to increased insulin levels in the plasma after glucose stimulation was next analyzed. Both plasma insulin and glucagon levels from blood samples obtained during IPGTT were measured (FIG. 6). It was found that insulin and glucagon levels were altered by administration of TBZ. Plasma insulin levels were, in general, greater following TBZ and glucose challenge relative to the vehicle treated controls. In four out of five experiments with different animals, the AUC INS with TBZ treatment was greater than two fold the AUC INS of control animals. Plasma glucagon levels were generally lower relative to controls following i.v. TBZ administration and glucose challenge. In three of five experiments, the AUG GCG in the presence of TBZ was 75%-85% less than the AUC GCG measured for the control animals. It was also noted that, prior to glucose challenge, the baseline plasma concentrations of glucagon were sometimes lower than controls, although these differences did not reach statistical significance.

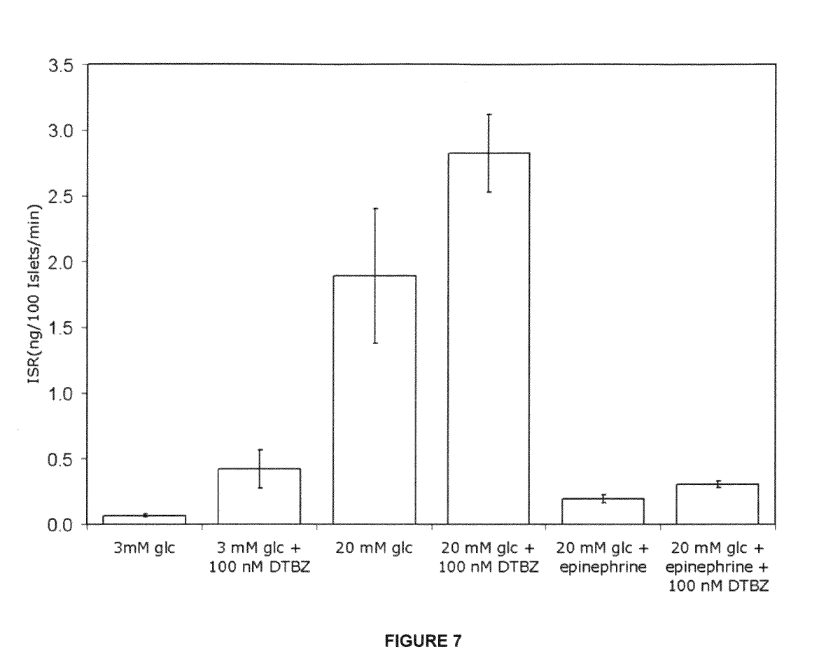

TBZ enhances insulin secretion in human cadaveric islets. Because VMAT2 is located throughout the CNS and glucose homeostasis is regulated by both the autonomic and sympathetic nervous system, whether TBZ was acting centrally and/or locally in islets was next considered. More particularly, because of their availability and clinical relevance, whether TBZ could enhance insulin secretion in purified human islet tissue ex vivo was tested. For these studies, clinical grade human islets that had not been utilized for transplantation were used. The islets were incubated in high and low glucose media with and without dihydrotetrabenazine (DTBZ). It was found that incubation of human islets in DTBZ significantly enhanced the amount of insulin secreted by islets in culture following stimulation by high concentrations of glucose (FIG. 7).

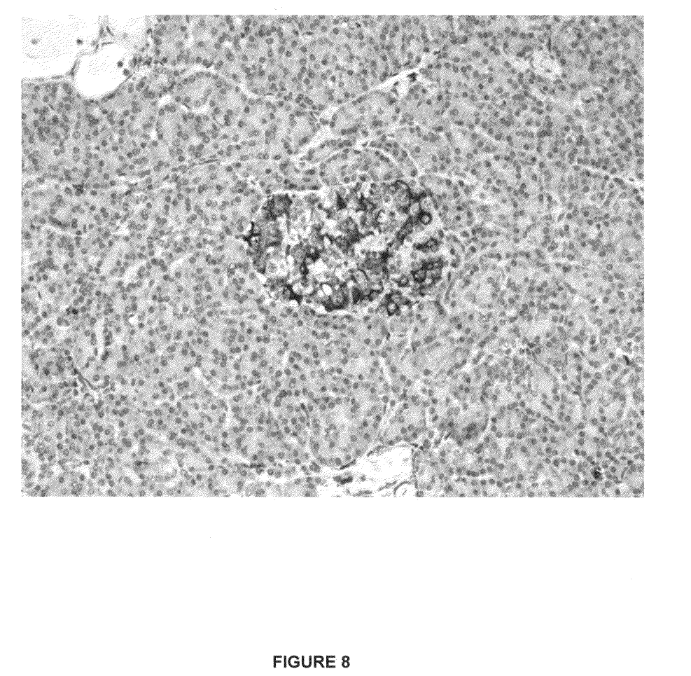

In control experiments, islets were incubated with epinephrine. As expected, epinephrine inhibited secretion of insulin in response to glucose stimulation. In the absence of high glucose stimulation, an increase in insulin secretion mediated by tetrabenazine was not observed (data not shown). Immunohistochemistry of pancreas sections confirmed that VMAT2 is localized to human islets (FIG. 8) and suggests that tetrabenazine mediates its effects on glucose metabolism directly by interfering with VMAT2-mediated monoamine transport within islet tissue.

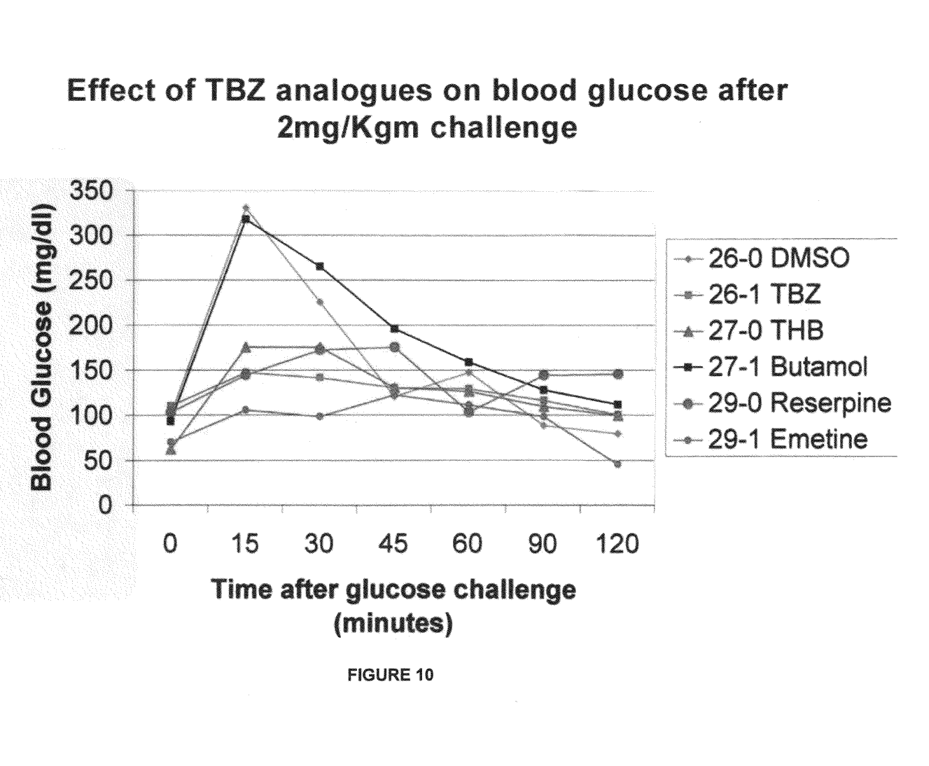

Glucose tolerance in Diabetic Lewis rats is also improved by THB, reserpine, and emetine. During IPGTT testing, blood glucose levels were returned to control or near normal levels at around sixty minutes following glucose challenge for those animals treated with an i.v. injection of TBZ, THB, butamol, reserpine, or emetine at dose of 2 mg/kg body weight (FIG. 10). Tetrabenazine, tetrahydroberberine (THB), reserpine, and emetine reduced the blood glucose excursion during an IPGTT. Butamol, however, did not reduce the blood glucose excursion during an IPGTT. Following glucose challenge, it was found that administration of TBZ, THB, butamol, reserpine, or emetine resulted in a smaller area under the curve in the IPGTT.

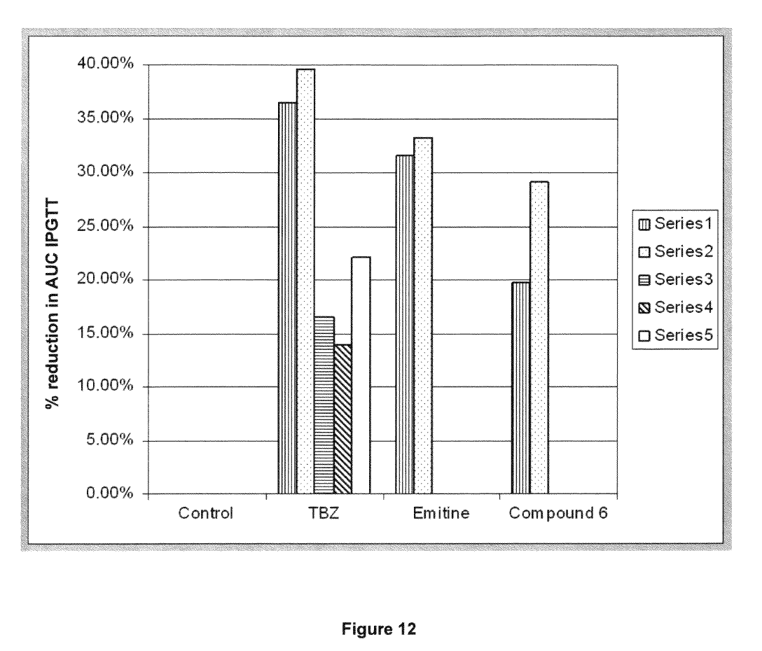

Glucose tolerance in Lewis rats is also improved by Compound 6. Lewis rats were selected for and subjected to IPGTT testing with and without a single dose of TBZ, emetine, and Compound 6 (2-3 mg/kg body weight) as previously described. As shown, TBZ, emetine, and Compound 6 consistently reduced the blood glucose excursion during an IPGTT, because these compounds consistently suppressed the area under the curve from IPGTT (FIG. 12).

Several previous studies have demonstrated a link between insulin secretion and dopamine. For example, it has been shown that treating Parkinson's patients with a dopamine precursor, L-DOPA, reduces insulin secretion in glucose tolerance tests. In rodent experiments, i.v. administration of L-DOPA has been shown to inhibit glucose-stimulated insulin secretion. Similarly, in culture, analogues of dopamine have been reported to inhibit glucose-stimulated insulin release by purified islets. More recently, it has been demonstrated that mouse β-cells (INS-1E cells), as well as purified rat and human islets, express the dopamine D2 receptor. In these cells and tissues, the D2 receptor was shown to co-localize with insulin in secretory granules. Both dopamine and the D2-like receptor agonist, quinpirole, inhibited glucose-stimulated insulin secretion when tested in primary rat β-cells, and pancreatic islets of rat, mouse, and human origin.

In the above example, it is shown that TBZ depletes the total dopamine content of the pancreas and enhances islet β-cell insulin secretion both in vivo and ex vivo. In light of the foregoing, the following model for the role of VMAT2 in islet function can be constructed. Dopamine, either produced in the exocrine pancreas or locally by β-cells, is transported and stored in insulin containing vesicles. In the presence of tetrabenazine, unsequestered dopamine is destroyed by monoamine oxygenases present in β-cells. Under normal glucose-stimulated insulin secretion, dopamine is also released with insulin and acts either in an autocrine or paracrine fashion to limit glucose-stimulated insulin secretion by other β-cells within the same islet or a distant islet.

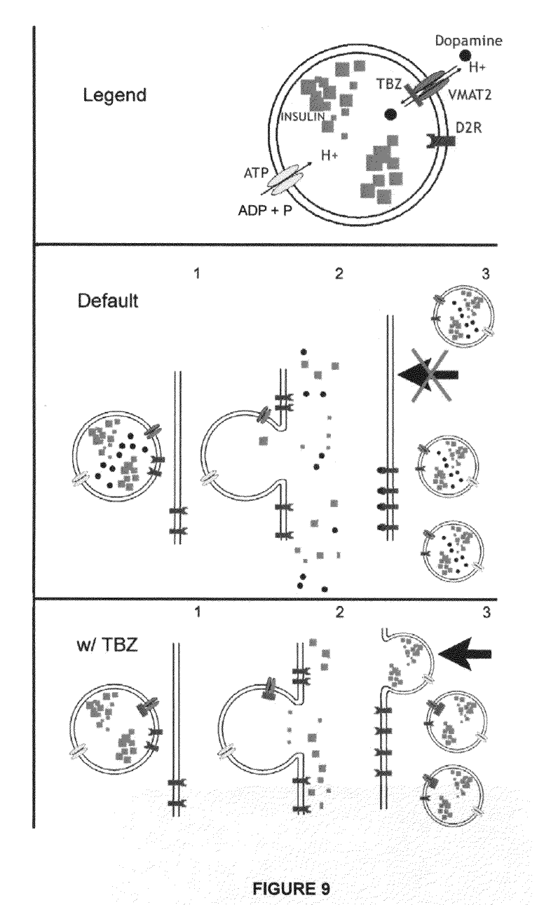

In the presence of tetrabenazine, this negative feedback loop is not present and dopamine is not released with insulin and other β-cells are left uninhibited (FIG. 9). Clearly, this model and the above observations must be interpreted carefully. Tetrabenazine has been used to treat movement disorders for over thirty years and glucose homeostasis related side effects have not been reported. Nevertheless, the above data argue that VMAT2 plays an important role in glucose homeostasis and constitutes a new target for intervention in (and treatment and/or prevention of) hyperglycemic disorders.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating or ameliorating the effects of diabetes comprising administering to a patient an effective amount of a vesicular monoamine transporter type 2 (VMAT2) antagonist.

2. A method for treating or preventing hyperglycemia, which comprises administering to the patient an effective amount of a vesicular monoamine transporter type 2 (VMAT2) antagonist.

3. The method according to any one of claim 1 or 2, wherein the VMAT2 antagonist is tetrabenazine (TBZ) or enantiomers, optical isomers, diastereomers, N-oxides, crystalline forms, hydrates, metabolites, or pharmaceutically acceptable salts thereof.

4. The method according to claim 3, wherein a metabolite of TBZ is dihydrotetrabenazine (DTBZ) or enantiomers, optical isomers, diastereomers, N-oxides, crystalline forms, hydrates, or pharmaceutically acceptable salts thereof.

5. The method according to any one of claim 1 or 2, wherein the VMAT2 antagonist is selected from the group consisting of tetrahydroberberine (THB), reserpine, emetine, Compound 6, and enantiomers, optical isomers, diastereomers, N-oxides, crystalline forms, hydrates, metabolites, pharmaceutically acceptable salts, and combinations thereof.

6. The method according to any one of claim 1 or 2, wherein the VMAT2 antagonist is administered intravenously.

7. The method according to claim 6, wherein a total of 3.3 mg of the VMAT2 antagonist is administered to the patient during a treatment course.

8. The method according to claim 7, wherein the VMAT2 antagonist is administered to the patient one to four times per day during the treatment course.

9. The method according to claim 7, wherein the VMAT2 antagonist is administered once per day during the treatment course.

10. The method according to claim 7, wherein the VMAT2 antagonist is administered twice per day during the treatment course.

11. The method according to any one of claim 1 or 2, wherein the effective amount is a dose sufficient to deplete monoamine levels from the patient's pancreas, but does not affect monoamine levels in the patient's brain.

12. The method according to claim 11, wherein the effective amount is between about 0.2 mg/kg body weight to about 5.0 mg/kg body weight of the VMAT2 antagonist.

13. The method according to claim 11, wherein the effective amount is about 0.5 to about 3.3 mg/kg body weight.

14. The method according to claim 11, wherein the effective amount is about 1.6 mg/kg body weight.

15. A method for treating diabetes comprising intravenously administering to a patient in need thereof about 1.6 mg/kg body weight of a vesicular monoamine transporter type 2 (VMAT2) antagonist selected from the group consisting of tetrabenazine (TBZ), dihydrotetrabenazine (DTBZ), and enantiomers, optical isomers, diastereomers, N-oxides, crystalline forms, hydrates, metabolites, pharmaceutically acceptable salts, and combinations thereof.

16. A method for treating diabetes comprising intravenously administering to a patient in need thereof about 2.0 mg/kg body weight of a vesicular monoamine transporter type 2 (VMAT2) antagonist selected from the group consisting of tetrahydroberberine (THB), reserpine, emetine, Compound 6, and enantiomers, optical isomers, diastereomers, N-oxides, crystalline forms, hydrates, metabolites, pharmaceutically acceptable salts, and combinations thereof.

17. The method according to claim 15 or 16, wherein a total of 3.3 mg of the VMAT2 antagonist is administered to the patient during a treatment course.

18. A method for treating or preventing hyperglycemia, which comprises intravenously administering to a patient in need thereof about 1.6 mg/kg body weight of a vesicular monoamine transporter type 2 (VMAT2) antagonist selected from the group consisting of tetrabenazine (TBZ), dihydrotetrabenazine (DTBZ), and enantiomers, optical isomers, diastereomers, N-oxides, crystalline forms, hydrates, metabolites, pharmaceutically acceptable salts, and combinations thereof.

19. A method for treating or preventing hyperglycemia, which comprises intravenously administering to a patient in need thereof about 2.0 mg/kg body weight of a vesicular monoamine transporter type 2 (VMAT2) antagonist selected from the group consisting of tetrahydroberberine (THB), reserpine, Compound 6, emetine, and enantiomers, optical isomers, diastereomers, N-oxides, crystalline forms, hydrates, metabolites, pharmaceutically acceptable salts, and combinations thereof.

20. The method according to any one of claim 18 or 19, wherein a total of 3.3 mg of the VMAT2 antagonist is administered to the patient during a treatment course.

21. A method for modulating monoamine levels in a patient in need of such modulation, which comprises administering to the patient an effective amount of a vesicular monoamine transporter type 2 (VMAT2) antagonist.

22. A method for modulating islet β-cell insulin secretion in a patient in need of such modulation, which comprises administering to the patient an effective amount of a vesicular monoamine transporter type 2 (VMAT2) antagonist.

23. A method for modulating insulin and glucagon levels in a patient in need of such modulation, which comprises administering to the patient an effective amount of a vesicular monoamine transporter type 2 (VMAT2) antagonist.

24. A method for regulating insulin production and glucose homeostasis in a patient in need thereof, which comprises administering to the patient an effective amount of a vesicular monoamine transporter type 2 (VMAT2) antagonist.

25. The method according to any one of claim 21, 22, 23, or 24, wherein the VMAT2 antagonist is tetrabenazine (TBZ) or enantiomers, optical isomers, diastereomers, N-oxides, crystalline forms, hydrates, metabolites, or pharmaceutically acceptable salts thereof.

26. The method according to claim 25, wherein a metabolite of TBZ is dihydrotetrabenazine (DTBZ) or enantiomers, optical isomers, diastereomers, N-oxides, crystalline forms, hydrates, or pharmaceutically acceptable salts thereof.

27. The method according to any one of claim 21, 22, 23, or 24, wherein the VMAT2 antagonist is selected from the group consisting of tetrahydroberberine (THB), reserpine, emetine, Compound 6, and enantiomers, optical isomers, diastereomers, N-oxides, crystalline forms, hydrates, metabolites, pharmaceutically acceptable salts thereof, and combinations thereof.

28. The method according to any one of claim 21, 22, 23, or 24, wherein the VMAT2 antagonist is administered intravenously.

29. The method according to claim 28, wherein a total of 3.3 mg of the VMAT2 antagonist is administered to the patient during a treatment course.

30. The method according to claim 29, wherein the VMAT2 antagonist is administered one to four times per day during the treatment course.

31. The method according to claim 29, wherein the VMAT2 antagonist is administered once per day during the treatment course.

32. The method according to claim 29, wherein the VMAT2 antagonist is administered twice per day during the treatment course.

33. The method according to any one of claim 21, 22, 23, or 24, wherein the effective amount is a dose sufficient to deplete monoamine levels from the patient's pancreas, but does not affect monoamine levels in the patient's brain.

34. The method according to claim 33, wherein the effective amount is between about 0.2 mg/kg body weight to about 5.0 mg/kg body weight of the VMAT2 antagonist.

35. The method according to claim 33, wherein the effective amount is about 0.5 to about 3.3 mg/kg body weight.

36. The method according to claim 33, wherein the effective amount is about 1.6 mg/kg body weight.

37. The method according to claim 21, wherein the monoamine is dopamine.

38. The method according to claim 22, wherein the modulating comprises increasing islet β-cell insulin secretion relative to a patient who is not administered the VMAT2 antagonist.

39. The method according to claim 23, wherein the modulating comprises increasing plasma insulin levels and decreasing plasma glucagon levels compared to a patient not treated with the VMAT2 antagonist.

40. A method for modulating glucose-stimulated insulin secretion in human islets comprising providing to the islets an amount of a vesicular monoamine transporter type 2 (VMAT2) antagonist that is effective to achieve the modulation.

41. The method according to claim 40, wherein the VMAT2 antagonist is tetrabenazine (TBZ) or enantiomers, optical isomers, diastereomers, N-oxides, crystalline forms, hydrates, metabolites, or pharmaceutically acceptable salts thereof.

42. The method according to claim 41, wherein a metabolite of TBZ is dihydrotetrabenazine (DTBZ) or enantiomers, optical isomers, diastereomers, N-oxides, crystalline forms, hydrates, or pharmaceutically acceptable salts thereof.

43. The method according to claim 40, wherein the VMAT2 antagonist is selected from the group consisting of tetrahydroberberine (THB), reserpine, emetine, Compound 6, and enantiomers, optical isomers, diastereomers, N-oxides, crystalline forms, hydrates, metabolites, pharmaceutically acceptable salts, and combinations thereof.

44. The method according to claim 40, wherein the modulating comprises increasing insulin secretion in the human islets compared to human islets not provided with the VMAT2 antagonist.


SEQUENCE LISTING (6 sequences)

SEQ ID NO: 1 - DNA, 21 bp, Artificial
  synthetic construct; cyclophilin A-5′
cttcgacatcacggctgatgg

SEQ ID NO: 2 - DNA, 20 bp, Artificial
  synthetic construct; cyclophilin A-3′
caggacctgtatgcttcagg

SEQ ID NO: 3 - DNA, 20 bp, Artificial
  synthetic construct; VMAT2-5′
gccctgcccatctggatgat

SEQ ID NO: 4 - DNA, 23 bp, Artificial
  synthetic construct; VMAT2-3′
ctttgcaatagcaccaccagcag

SEQ ID NO: 5 - DNA, 18 bp, Artificial
  synthetic construct; rINS1/2-5′
cccaggcttttgtcaaac

SEQ ID NO: 6 - DNA, 18 bp, Artificial
  synthetic construct; rINS1/2-3′
cttgcgggtcctccactt